(19) United States
(12) Patent Application Publication
(10) Pub. No.: US 2025/0049830 A1
(43) Pub. Date: Feb. 13, 2025

(54) PHARMACEUTICAL COMPOSITION FOR PREVENTING OR TREATING CANCER, COMPRISING SGLT-2 INHIBITOR AND GOSSYPOL

(71) Applicant: HAIM BIO., LTD., Seongbuk-gu Seoul (KR)

(72) Inventors: Yong Baeg Kim, Gangseo-gu Seoul (KR); Sun Young Park, Songi-ro Songpagu Seoul (KR); Se Young Jeong, Dongjak-gu Seoul (KR); Hong Yeoul Kim, Seongdong-gu Seoul (KR)

(73) Assignee: HAIM BIO., LTD., Seongbuk-gu Seoul (KR)

(21) Appl. No.: 18/722,680

(22) PCT Filed: Dec. 30, 2020
(86) PCT No.: PCT/KR2020/019368

§ 371 Date: Jun. 21, 2024

(30) Foreign Application Priority Data
Dec. 30, 2019 (KR) 10-2019-0177439

(51) Int. Cl. A61K 31/7042; A61K 31/12; A61K 31/155; A61P 35/00; A61P 35/04

(52) U.S. Cl. CPC A61K 31/7042; A61K 31/12; A61K 31/155; A61P 35/00; A61P 35/04

(57) ABSTRACT

The present invention relates to a pharmaceutical composition for co-administration for effectively preventing or treating cancer. The pharmaceutical composition of the present invention is applicable to various cancer types, and the effect of killing cancer cells and/or cancer stem cells is significantly improved when the active ingredients of the composition are administered in combination compared to when the active ingredients are administered alone. Thus, the pharmaceutical composition is expected to be widely used for cancer treatment in the medical and health fields.

### TECHNICAL FIELD

The present invention relates to a pharmaceutical composition capable of effectively preventing or treating cancer.

### BACKGROUND ART

Cancer is one of incurable diseases to be resolved by humanity and a huge amount of capital has been invested in development for treating the cancer around the world. In South Korea, cancer is the number one cause of death by disease, with more than about 100,000 people diagnosed with cancer each year and more than about 60,000 people dying from cancer each year.

Carcinogens that cause cancer include smoking, ultraviolet rays, chemicals, food, and other environmental factors. However, because the causes of cancer are diverse, it is difficult to develop therapeutic agents, and the effectiveness of the therapeutic agents also varies depending on the area where cancer occurs. Substances that are currently used as therapeutic agents have significant toxicity and do not selectively remove only cancer cells, and thus there is an urgent need for the development of less toxic and effective anticancer agents to not only treat cancer after its occurrence but also prevent the occurrence of cancer. Although there has been rapid progress in cancer diagnosis and treatment over the past 10 years, the mortality rate due to cancer is still high.

The present invention has been made in order to solve the above-described problems and relates to a pharmaceutical composition for treating cancer containing a SGLT2 (sodium-glucose cotransporter-2) inhibitor as an active ingredient. SGLT2 inhibitors, also known as gliflozin, are drugs that lower blood sugar by inhibiting the reabsorption of glucose in the kidneys. They work by inhibiting sodium/glucose cotransporter-2 (SGLT2), and are mainly used for the treatment of type II diabetes mellitus (T2DM).

Meanwhile, gossypol as a polyphenolic compound is a phenol derivative that is contained in cotton plants in large amounts, and it was recently reported that gossypol has a significant effect on the inhibition of cancer cell growth (U.S. Pat. No. 6,114,397). However, it is still difficult to effectively inhibit cancer cell growth by administration of gossypol alone. The present invention has found that the effect of killing cancer cells is significantly improved when an SGLT-2 inhibitor and gossypol are administered in combination compared to when the SGLT-2 inhibitor or gossypol is administered alone. The pharmaceutical composition of the present invention, which contains the SGLT-2 inhibitor and gossypol as active ingredients, has a significant cancer therapeutic effect on various cancer types, and is therefore expected to be widely used as a new alternative to anticancer therapy in the medical and health fields.

### DISCLOSURE

### Technical Problem

One object of the present invention is to provide a pharmaceutical composition that can more effectively prevent or treat cancer by administering two or more active ingredients in combination to cancer patients.

Another object of the present invention is to provide a pharmaceutical composition that can inhibit cancer metastasis by administering two or more active ingredients in combination to cancer patients.

Still another object of the present invention is to provide a food composition that can prevent or alleviate cancer by administering two or more active ingredients in combination to cancer patients.

However, objects to be achieved by the present invention are not limited to the objects mentioned above, and other objects not mentioned above can be clearly understood by those skilled in the art from the following description.

### Technical Solution

Hereinafter, various embodiments described herein will be described with reference to figures. In the following description, numerous specific details are set forth, such as specific configurations, compositions, and processes, etc., in order to provide a thorough understanding of the present invention. However, certain embodiments may be practiced without one or more of these specific details, or in combination with other known methods and configurations. In other instances, known processes and preparation techniques have not been described in particular detail in order to not unnecessarily obscure the present invention. Reference throughout this specification to “one embodiment” or “an embodiment” means that a particular feature, configuration, composition, or characteristic described in connection with the embodiment is included in at least one embodiment of the present invention. Thus, the appearances of the phrase “in one embodiment” or “an embodiment” in various places throughout this specification are not necessarily referring to the same embodiment of the present invention. Additionally, the particular features, configurations, compositions, or characteristics may be combined in any suitable manner in one or more embodiments.

Unless otherwise stated in the specification, all the scientific and technical terms used in the specification have the same meanings as commonly understood by those skilled in the art to which the present invention pertains.

According to one embodiment of the present invention, the present invention is directed to a pharmaceutical composition for preventing or treating cancer containing a sodium glucose cotransporter-2 inhibitor or a pharmaceutically acceptable salt thereof as an active ingredient.

In one embodiment of the present invention, “cancer” is characterized by unregulated cell growth. This abnormal cell growth results in the formation of a cell mass called a tumor, which infiltrates into surrounding tissues and, in severe cases, metastasize into other organs of the body. Academically, cancer is also called neoplasia. Cancer is an intractable chronic disease that is not fundamentally cured in many cases even if it is treated by surgery, radiotherapy, and chemotherapy, causes pain to the patient, and ultimately leads to death. Cancer is caused by various factors which are divided into internal factors and external factors. Although a mechanism by which normal cells are transformed into cancer cells has not been clearly found, it is known that a significant number of cancers are caused by external factors such as environmental factors. The internal factors include genetic factors, immunological factors, and the like, and the external factors include chemical substances, radiations, viruses, and the like. Genes involved in cancer development include oncogenes and tumor suppressor genes, and cancer develops when the balance between these genes is broken by the above-described internal or external factors.

The pharmaceutical composition of the present invention is used to prevent or treat the above-described cancer, and may be applied to, without limitation, solid tumors such as fibrosarcoma, myxosarcoma, liposarcoma, chondrosarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, anal cancer, colon cancer, colorectal cancer, colon cancer, small intestine cancer, kidney cancer, bladder cancer, pancreatic cancer, osteosarcoma, breast cancer, ovarian cancer, prostate cancer, esophageal cancer, gallbladder cancer, thyroid cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, skin cancer, melanoma, neuroblastoma, and retinoblastoma; blood-borne cancers such as lymphoma, acute lymphoblastic leukemia (“ALL”), acute lymphoblastic B-cell leukemia, acute lymphoblastic T-cell leukemia, acute myeloblastic leukemia (“AML”), acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), hairy cell leukemia, and multiple myeloma; acute and chronic leukemias such as lymphoblastic, myelogenous, lymphocytic, myelocytic leukemias; lymphomas such as Hodgkin's disease, non-Hodgkin's lymphoma, multiple myeloma, Waldenstrom's macroglobulinemia, heavy chain disease, and polycythemia vera; CNS and brain cancers such as glioma, pilocytic astrocytoma, anaplastic astrocytoma, glioblastoma multiforme, medulloblastoma, craniopharyngioma, ependymoma, pinealoma, hemangioblastoma, acoustic neuroma, oligodendroglioma, meningioma, vestibular schwannoma, adenoma, brain tumor, metastatic brain tumor, meningioma, spinal tumor, and medulloblastoma.

In one embodiment of the present invention, “cancer stem cells” refers to cancer cells in a comprehensive sense that have self-renewal or differentiation ability, which is the unique ability of stem cells. For example, cancer stem cells may include a spherical cancer cell population or a cancer tissue having an unclear shape and poor prognosis. In the normal tumor growth conditions of cancer stem cells (the “normal tumor growth conditions” refers to a state in which a nutrient (glucose) required for cell growth is sufficient and conditions for tumor microenvironment growth are abundant, and thus there is no cell stress), the cancer stem cells may proliferate at a slow rate, unlike common cancer cells, or may be maintained in a dormant state, and thus may have resistance to anticancer agents. For example, expression of transcription regulators such as PGC-1a may be controlled, unlike that in normal tumor cells, and thus the function of major metabolism regulatory substances therein may differ from that in common cancer cells. Thus, “cancer stem cells” generally refers to cells that acquire resistance to apoptosis in a nutrient-deficient state through this different metabolism regulatory ability and the regulation of cell signaling systems mechanistically linked thereto, and have invasive and/or metastatic potential. However, the cancer stem cells are not limited thereto and may include any cells that may differentiate into common cancer cells. Therefore, “cancer” in the present invention is meant to include cancer stem cells.

In one embodiment of the present invention, “sodium-glucose cotransporter-2 (SGLT-2)” refers to a protein encoded by the human SLC5A2 (solute carrier family 5 (sodium-glucose cotransporter)) gene. It is known that cancer cells require large amounts of glucose to survive and grow, and that some cancers rely on SGLT to utilize glucose (Sci Transl Med. 2018 Nov. 14; 10 (467)). Therefore, SGLT2 can be used as a biomarker for prediction and diagnosis of cancer. Recently, it was found that SGLT-2 inhibitors have a therapeutic effect even for patients with non-diabetic heart failure related to kidney disease. Thus, the American College of Cardiology and the European Society of Cardiology are planning to recommend that SGLT-2 inhibitors be used first in high-risk groups with cardiovascular risk factors. In addition, indications of anti-diabetic agents of the SGLT-2 inhibitor class have been expanded to kidney diseases.

The sodium-glucose cotransporter-2 inhibitor of the present invention may comprise any one or more selected from the group consisting of canagliflozin, dapagliflozin, empagliflozin, sotagliflozin, ipragliflozin, luseogliflozin, ertugliflozin, tofogliflozin, and remogliflozin. Preferably, the sodium-glucose cotransporter-2 inhibitor may comprise any one or more selected from the group consisting of dapagliflozin, empagliflozin, and canagliflozin. More preferably, the sodium-glucose cotransporter-2 inhibitor may be canagliflozin (Ann Pharmacother. 2019 December; 53 (12): 1227-1237).

The “dapagliflozin”, a drug of the SGLT-2 inhibitor class, is used to treat type-2 diabetes and is sold under the brand name Farxiga. The drug inhibits sodium-glucose cotransporter subtype 2 (SGLT2), which functions to reabsorb glucose in the kidneys. This mechanism works to lower blood sugar levels through urine.

The “empagliflozin”, a drug of the SGLT-2 inhibitor class, is used to treat type-2 diabetes and is sold under the brand name Jardiance. Meanwhile, it is known to be less effective in treating diabetes than metformin. The empagliflozin is a compound with the formula C₂₃H₂₇ClO₇ and has the characteristic of reducing blood sugar by inhibiting the SGLT-2 receptor, blocking glucose reabsorption in the kidneys and excreting sugar through urine.

In addition, the “canagliflozin” refers to a (1S)-1,5-anhydro-1-C-(3-{[5-(4-fluorophenyl)thiophene-2-yl]methyl]}-4-methylphenyl)-D-glucitol compound and is widely known as an anti-diabetic drug that helps control blood sugar, like biguanide drugs such as metformin. Canagliflozin is a substance called “flozin” isolated from apple peels and was discovered in the past, but it was so toxic that it could not be used as a medicine. However, since canagliflozin was found to act selectively only against SGLT-2, and to exhibit better effects in patients with type 2 diabetes not controlled by metformin, it is widely used as an anti-diabetic drug (J Korean Diabetes. 2014 September; 15 (3): 146-150). Canagliflozin is also known to inhibit the growth of some cancers (Mol Metab. 2016 Aug. 26; 5 (10): 1048-1056). Canagliflozin was recently approved by the FDA to be used as a therapeutic agent for lowering the risk of end-stage renal disease and death from kidney disease or cardiovascular disease in adult patients with type-2 diabetes and chronic kidney disease.

The pharmaceutically acceptable salts may include acid or base addition salts and their stereochemical isomers. For example, the compound may be in the form of an organic acid or inorganic acid addition salt. The salts include, but are not particularly limited to, any salts that retain the activity of their parent compounds when administered to a patient and have a desirable effect in the patient. These salts may include inorganic salts and organic salts, for example, salts of acetic acid, nitric acid, aspartic acid, sulfonic acid, sulfuric acid, maleic acid, glutamic acid, formic acid, succinic acid, phosphoric acid, phthalic acid, tannic acid, tartaric acid, hydrobromic acid, propionic acid, benzenesulfonic acid, benzoic acid, stearic acid, lactic acid, bicarbonic acid, bisulfuric acid, bitartaric acid, oxalic acid, butyric acid, calcium edetate, carbonic acid, chlorobenzoic acid, citric acid, edetic acid, toluenesulfonic acid, fumaric acid, gluceptic acid, esylic acid, pamoic acid, gluconic acid, methylnitric acid, malonic acid, hydrochloric acid, hydroiodoic acid, hydroxynaphtholic acid, isethionic acid, lactobionic acid, mandelic acid, mucous acid, naphthoic acid, muconic acid, p-nitromethane sulfonic acid, hexamic acid, pantothenic acid, monohydrogen phosphate, dihydrogen phosphate, salicylic acid, sulfamic acid, sulfanilic acid, methanesulfonic acid, etc. Base addition salts may include salts of alkali or alkaline earth metals, such as ammonium, lithium, sodium, potassium, magnesium, or calcium; salts with organic bases, for example, benzathine, N-methyl-D-glucamine, or hydrabamine salts; and salts with amino acids such as arginine or lysine. In addition, these salts may be converted to a free form by treatment with an appropriate base or acid.

According to another embodiment of the present invention, the present invention is directed to a pharmaceutical composition for preventing or treating cancer containing, as an active ingredient, any one or more selected from the group consisting of a sodium glucose cotransporter-2 (SGLT-2) inhibitor, and gossypol, or derivatives or pharmaceutically acceptable salts thereof.

In one embodiment of the present invention, “gossypol” is a kind of polyphenolic compound contained in the separable pigmented lines of the seeds, leaves, stems and roots of some of plants belonging to the genus Gossypium of the family Malvaceae, and is also called polyphenolic gossypol or cottonseed pigment. It renders plants resistant to pests. It was reported that when gossypol was added to poultry feed, the feed utilization and the egg productivity were reduced and the yolk decolorization of stored eggs occurred. On the other hand, ruminant livestock inactivates gossypol by fermentation. Free gossypol is physiologically toxic, whereas bound gossypol is inactive. The non-protein components of cottonseeds also bind to gossypol to form non-soluble and/or non-digestible complexes. This binding detoxifies gossypol in cottonseed meal, but reduces protein and biological values. When iron is added to free gossypol at a ratio of 2:1 or 3:1, it can effectively reduce the toxicity of gossypol in the liver. In China, it was found that this gossypol inhibits male sperm function. Thus, the gossypol has been studied for use as male oral contraceptives. Gossypol in the present invention is meant to include all of (+)-gossypol, (−)-gossypol, (+) gossypol, (−) gossypol, or gossypol obtained by linking them, or gossypol derivatives such as hemigossypol or apogossypol.

In a preferred example of the present invention, the pharmaceutical composition may contain at least two of an SGLT-2 inhibitor, gossypol, or pharmaceutically acceptable salts thereof.

In a preferred example of the present invention, the pharmaceutical composition may contain, as the SGLT-2 inhibitor, any one or more selected from the group consisting of canagliflozin, dapagliflozin, empagliflozin, sotagliflozin, ipragliflozin, luseogliflozin, ertugliflozin, tofogliflozin, and remogliflozin. Preferably, the pharmaceutical composition may contain any one or more selected from the group consisting of dapagliflozin, empagliflozin, and canagliflozin. More preferably, the pharmaceutical composition may contain canagliflozin, without being limited thereto.

The pharmaceutical composition of the present invention is characterized by exhibiting a significantly good effect of preventing or treating cancer cells by administering the active ingredients in combination compared to when administering the active ingredients alone.

According to another embodiment of the present invention, the present invention is directed to a pharmaceutical composition further containing a biguanide-based compound as an active ingredient, in addition to the pharmaceutical composition for preventing or treating cancer containing a sodium glucose cotransporter-2 inhibitor or a pharmaceutically acceptable salt thereof as an active ingredient.

In one embodiment of the present invention, the “biguanide-based compound” refers to a compound for treatment of diabetes that helps the human body respond to insulin by gradually sending glucose stored in the liver to the blood. It is known that biguanide-based drugs act as resistance improvers to maintain blood sugar levels at a constant level rather than rapidly lowering blood sugar levels. The biguanide-based compound is mainly used as a drug to treat patients with type 2 diabetes, and the most widely used biguanide-based compound is metformin. The drug is also known to be useful in controlling fasting blood sugar because it inhibits hepatic gluconeogenesis. It has recently been proven that biguanide-based drugs have the effect of inhibiting cancer growth by affecting energy metabolism.

In a preferred example of the present invention, the pharmaceutical composition may contain a biguanide-based compound. Preferably, the pharmaceutical composition may contain any one or more selected from the group consisting of metformin, phenformin, and buformine. More preferably, the pharmaceutical composition may contain phenformin. However, the biguanide-based compound is not limited thereto and may be any biguanide-based compound or derivative thereof that induces a nutrient deficiency-like state by interfering with intracellular energy production.

According to another embodiment of the present invention, the present invention is directed to a pharmaceutical composition for preventing or treating cancer containing, as an active ingredient, any one or more selected from the group consisting of a sodium-glucose cotransporter-2 (SGLT-2) inhibitor, gossypol, a biguanide-based compound, and pharmaceutically acceptable salts.

According to another embodiment of the present invention, the present invention is directed to a pharmaceutical composition for inhibiting cancer metastasis containing, as an active ingredient, any one or more selected from the group consisting of a sodium-glucose cotransporter-2 (SGLT-2) inhibitor, gossypol, a biguanide-based compound, and pharmaceutically acceptable salts.

A disease to be prevented or treated using the pharmaceutical composition of the present invention may be cancer that has occurred or is likely to occur in a subject.

In the present invention, the “subject” refers to mammals including humans. For example, the subject may be selected from the group consisting of humans, rats, mice, guinea pigs, hamsters, rabbits, monkeys, dogs, cats, cows, horses, pigs, sheep, and goats. Preferably, the subject may be a human, without being limited thereto.

In one embodiment of the present invention, “prevention” may include, without limitation, any action of blocking, inhibiting or delaying cancer symptoms using the pharmaceutical composition of the present invention.

In one embodiment of the present invention, “treatment” means a series of actions performed to alleviate or/and ameliorate a target disease. For the purpose of the present invention, treatment may include, without limitation, any action that alleviates or beneficially changes cancer symptoms by administering the pharmaceutical composition of the present invention.

The pharmaceutical composition of the present invention may be used to effectively treat not only cancer stem cells but also common cancer cells by co-administration.

In one embodiment of the present invention, the “pharmaceutical composition” refers to a composition to be administered for a specific purpose. For the purpose of the present invention, the pharmaceutical composition of the present invention is for preventing or treating cancer, and may contain a compound involved therein and a pharmaceutically acceptable carrier, excipient, or diluent. In addition, the pharmaceutical composition according to the present invention contain the active ingredient of the present invention in an amount of 0.1 to 50 wt % based on the total weight of the composition.

In the present invention, the pharmaceutical composition may be in the form of capsules, tablets, granules, injections, ointments, powders, or beverages, and the pharmaceutical composition may be intended for administration to a human subject.

For use, the pharmaceutical composition of the present invention may be formulated in the form of oral preparations such as powders, granules, capsules, tablets, and aqueous suspensions, preparations for external use, suppositories, and sterile injectable solutions, according to the respective conventional methods, without being limited thereto. The pharmaceutical composition of the present invention may further contain pharmaceutically acceptable carriers. As the pharmaceutically acceptable carriers, a binder, a lubricant, a disintegrant, an excipient, a solubilizer, a dispersant, a stabilizer, a suspending agent, a colorant, a flavoring agent, and the like may be used for oral administration; a buffer, a preservative, a pain-relieving agent, a solubilizer, an isotonic agent, a stabilizer, and the like may be used for injection; and a base, an excipient, a lubricant, a preservative, and the like may be used for topical administration. The pharmaceutical composition of the present invention may be prepared in various dosage forms by being mixed with the pharmaceutically acceptable carriers described above. For example, for oral administration, the pharmaceutical composition may be formulated in the form of tablets, troches, capsules, elixirs, suspensions, syrups, wafers, or the like. For injection, the pharmaceutical composition may be formulated in the form of unit dosage ampoules or in multiple-dosage forms. In addition, the pharmaceutical composition may be formulated into solutions, suspensions, tablets, capsules, sustained-release preparations, or the like.

Meanwhile, examples of carriers, excipients and diluents suitable for formulation include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil. In addition, the pharmaceutical composition of the present disclosure may further contain a filler, an anticoagulant, a lubricant, a wetting agent, a fragrance, an emulsifier, a preservative, or the like.

The routes of administration of the pharmaceutical composition according to the present invention include, but are not limited to, oral, intravenous, intramuscular, intra-arterial, intramedullary, intradural, intracardiac, transdermal, subcutaneous, intraperitoneal, intranasal, gastrointestinal, topical, sublingual and intrarectal routes. Oral or parenteral administration is preferred.

In one embodiment of the present invention, “administration” means introducing the composition of the present invention into a patient by any suitable method. The composition of the present invention may be administered by any general route, as long as it can reach a target tissue. Examples of the administration route include, but are not limited to, oral administration, intraperitoneal administration, intravenous administration, intramuscular administration, subcutaneous administration, intradermal administration, intranasal administration, intrapulmonary administration, intrarectal administration, intracavitary administration, intraperitoneal administration, and intradural administration. In the present invention, the effective amount may be adjusted depending on various factors, including the type of disease, the severity of the disease, the types and contents of the active ingredient and other ingredients contained in the composition, the type of formulation, the patient's age, body weight, general health status, sex and diet, the time of administration, the route of administration, the secretion rate of the composition, the duration of treatment, and concurrently used drugs. For adults, the pharmaceutical composition for treatment may be administered into the body in an amount of 50 ml to 500 ml at a time, the compound may be administered at a dose of 0.1 ng/kg to 10 mg/kg, and a monoclonal antibody may be administered at a dose of 0.1 ng/kg to 10 mg/kg. Administration may be performed once to 12 times a day, and when administration is performed 12 times a day, administration may be performed once every 2 hours. In addition, the pharmaceutical composition of the present invention may be administered alone or in combination with other therapies known in the art, such as chemotherapy, radiotherapy, and surgery, for the treatment of the cancer stem cells of interest. The pharmaceutical composition of the present invention may also be administered in combination with other treatments designed to enhance immune responses, e.g., by co-administration with adjuvants or cytokines (or nucleic acids encoding cytokines), as is well known in the art. Other standard delivery methods, e.g., biolistic transfer or ex vivo treatment, may also be used. In ex vivo treatment, antigen presenting cells (APCs), dendritic cells, peripheral blood mononuclear cells, or bone marrow cells may be obtained from a patient or an appropriate donor and activated ex vivo with the pharmaceutical composition of the present invention, and then administered to the patient.

In the present invention, the “parenteral” includes subcutaneous, transdermal, intravenous, intramuscular, intra-articular, intra-synovial, intrasternal, intradural, intra-lesional and intra-cranial injection or infusion techniques. The pharmaceutical composition of the present invention may also be administered as suppositories for intrarectal administration.

The dose of the pharmaceutical composition of the present invention may vary depending on a variety of factors, including the activity of a specific compound used, the patient's age, body weight, general health status, sex and diet, the time of administration, the route of administration, the rate of excretion, drug combination, and the severity of a specific disease to be prevented or treated. Although the dose of the pharmaceutical composition may vary depending on the patient's condition and body weight, the severity of disease, the drug form, and the route and duration of administration, it may be appropriately selected by those skilled in the art, and the pharmaceutical composition may be administered at a dose of 0.0001 to 50 mg/kg/day or 0.001 to 50 mg/kg/day. The pharmaceutical composition may be administered once a day or several times a day. The dose is not intended to limit the scope of the present invention in any way. The pharmaceutical composition according to the present invention may be formulated in the form of pills, sugar-coated tablets, capsules, liquids, gels, syrups, slurries, or suspensions.

According to another embodiment of the present invention, the present invention is directed to a food composition for preventing or ameliorating cancer containing, as an active ingredient, any one or more selected from the group consisting of a sodium-glucose cotransporter-2 (SGLT-2) inhibitor, gossypol, a biguanide-based compound, and pharmaceutically acceptable salts thereof.

In one embodiment of the present invention, the “food composition” refers to a composition that is used in various ways to prevent or ameliorate the indications targeted by the present invention. The food composition containing the composition of the present invention as an active ingredient may be prepared as various foods, for example, beverages, gums, teas, vitamin complexes, powders, granules, tablets, capsules, confectionery, cakes, bread, and the like. Since the food composition of the present invention is an improved food composition containing existing food ingredients with little toxicity and side effects, it may be used with confidence even when it is administered for a long period of time for preventive purposes. When the composition of the present invention is contained in the food composition, it may be added in an amount of 0.1 to 100 wt % based on the total weight. When the food composition is prepared as a beverage, there is no particular limitation, except that the beverage contains the food composition at the indicated percentage. The beverage may additionally contain various flavorings or natural carbohydrates, like conventional beverages. Examples of the natural carbohydrates include monosaccharides such as glucose, disaccharides such as fructose, polysaccharides such as sucrose, conventional sugars such as dextrin and cyclodextrin, and sugar alcohols such as xylitol, sorbitol, and erythritol. Examples of the flavorings include natural flavorings (thaumatin, stevia extracts, such as rebaudioside A, glycyrrhizin, etc.) and synthetic flavorings (saccharin, aspartame, etc.). In addition, the food composition of the present invention may contain various nutrients, vitamins, minerals (electrolytes), flavorings such as synthetic flavorings and natural flavorings, colorants, pectic acid and its salt, alginic acid and its salt, organic acids, protective colloidal thickeners, pH adjusting agents, stabilizers, preservatives, glycerin, alcohol, carbonizing agents that are used in carbonated beverages, etc. Such components may be used individually or in combination. Although the content of these additives is generally selected in the range of 0.1 to about 100 parts by weight based on 100 parts by weight of the food composition of the present invention, without being limited thereto.

According to another embodiment of the present invention, the present invention is directed to a method for preventing or treating cancer, comprising a step of administering to a subject in need thereof a pharmaceutically effective amount of any one or more selected from the group consisting of a sodium-glucose cotransporter-2 (SGLT-2) inhibitor, gossypol, a biguanide-based compound, and pharmaceutically acceptable salts thereof.

According to another embodiment of the present invention, the present invention is directed to a method for inhibiting cancer metastasis, comprising a step of administering to a subject in need thereof a pharmaceutically effective amount of any one or more selected from the group consisting of a sodium-glucose cotransporter-2 (SGLT-2) inhibitor, gossypol, a biguanide-based compound, and pharmaceutically acceptable salts thereof.

In the present invention, the “administration” means providing a given compound of the present invention to a subject by any suitable method.

In the present invention, the “subject” in need thereof may include both mammals and non-mammals. Here, examples of the mammals include, but are not limited to, humans, non-human primates such as chimpanzees, other ape or monkey species; livestock animals such as cattle, horses, sheep, goats, and pigs; domesticated animals such as rabbits, dogs or cats; laboratory animals, for example, rodents such as rats, mice, or guinea pigs. In addition, examples of the non-mammals in the present invention include, but are not limited to, birds or fish.

In the present invention, the formulation of the compound that is administered as described above is not particularly limited, and may be administered as solid form preparations, liquid form preparations, or aerosol preparations for inhalation. Specifically, the compound may be administered as solid form preparations which are intended to be converted, shortly before use, to liquid form preparations for oral or parenteral administration. For example, the compound may be formulated and administered as oral dosage forms, including powders, granules, capsules, tablets, and aqueous suspensions, preparations for external use, suppositories, and sterile injectable solutions, without being limited thereto.

In addition, in the present invention, pharmaceutically acceptable carriers may be additionally administered along with the compound of the present invention during the administration. Here, as the pharmaceutically acceptable carriers, a binder, a lubricant, a disintegrant, an excipient, a solubilizer, a dispersant, a stabilizer, a suspending agent, a colorant, a flavoring agent, and the like may be used for oral administration; a buffer, a preservative, a pain-relieving agent, a solubilizer, an isotonic agent, a stabilizer, and the like may be used for injection; and a base, an excipient, a lubricant, a preservative, and the like may be used for topical administration. In addition, the compound of the present invention may be prepared in various dosage forms by being mixed with the pharmaceutically acceptable carriers described above. For example, for oral administration, the compound may be formulated in the form of tablets, troches, capsules, elixirs, suspensions, syrups, wafers, or the like. For injection, the compound may be formulated in the form of unit dosage ampoules or in multiple-dosage forms. In addition, the compound may be formulated into solutions, suspensions, tablets, capsules, sustained-release preparations, or the like.

Meanwhile, examples of carriers, excipients and diluents suitable for formulation include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil. In addition, the composition of the present disclosure may further contain a filler, an anticoagulant, a lubricant, a wetting agent, a fragrance, an emulsifier, a preservative, or the like.

The routes of administration of the compound according to the present invention include, but are not limited to, oral, intravenous, intramuscular, intra-arterial, intramedullary, intradural, intracardiac, transdermal, subcutaneous, intraperitoneal, intranasal, gastrointestinal, topical, sublingual and intrarectal routes. Oral or parenteral administration is preferred.

In the present invention, the “parenteral” includes subcutaneous, transdermal, intravenous, intramuscular, intra-articular, intra-synovial, intrasternal, intradural, intra-lesional and intra-cranial injection or infusion techniques. The pharmaceutical composition of the present invention may also be administered as suppositories for intrarectal administration.

In the present invention, the term “pharmaceutically effective amount” refers to a sufficient amount of an agent to provide a desired biological result. Said result may be reduction and/or alleviation of a sign, symptom, or cause of a disease, or any other desired alteration of a biological system. For example, an “effective amount” for therapeutic uses is the amount of the compound disclosed in the present invention, which is required to provide a clinically significant reduction in the disease. An appropriate “effective amount” in any individual case may be determined by one of ordinary skill in the art using routine experimentation. Thus, the expression “effective amount” generally refers to an amount in which an active substance has a therapeutic effect. In the case of the present invention, the active substance serves as both an inhibitor of the growth of cancer tumor spheroids and an agent for preventing, ameliorating or treating cancer.

The dose of the compound of the present invention may vary depending on a variety of factors, including the activity of a specific compound used, the patient's age, body weight, general health status, sex and diet, the time of administration, the route of administration, the rate of excretion, drug combination, and the severity of a specific disease to be prevented or treated. Although the dose of the compound may vary depending on the patient's condition and body weight, the severity of disease, the drug form, and the route and duration of administration, it may be appropriately selected by those skilled in the art, and may be administered at a dose of 0.0001 to 100 mg/kg/day or 0.001 to 100 mg/kg/day. The compound may be administered once a day or several times a day. The dose is not intended to limit the scope of the present invention in any way. The compound according to the present invention may be formulated in the form of pills, sugar-coated tablets, capsules, liquids, gels, syrups, slurries, or suspensions.

The compound of the present invention may be used alone or in combination with surgery, radiotherapy, hormone therapy, chemotherapy, and methods that use biological response modifiers.

In addition, the compound of the present invention may be used in combination with other anticancer drug. Here, the anticancer drug may be at least one selected from the group consisting of nitrogen mustard, imatinib, oxaliplatin, rituximab, erlotinib, neratinib, lapatinib, gefitinib, vandetanib, nirotinib, semasanib, bosutinib, axitinib, cediranib, lestaurtinib, trastuzumab, gefitinib, bortezomib, sunitinib, carboplatin, sorafenib, bevacizumab, cisplatin, cetuximab, viscum album, asparaginase, tretinoin, hydroxycarbamide, dasatinib, estramustine, gemtuzumab ozogamicin, ibritumomab tiuxetan, heptaplatin, methyl aminolevulinic acid, amsacrine, alemtuzumab, procarbazine, alprostadil, holmium nitrate chitosan, gemcitabine, doxifluridine, pemetrexed, tegafur, capecitabine, gimeracin, oteracil, azacitidine, methotrexate, uracil, cytarabine, fluorouracil, fludarabine, enocitabine, flutamide, capecitabine, decitabine, mercaptopurine, thioguanine, cladribine, carmophor, raltitrexed, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, pepromycin, temsirolimus, temozolomide, busulfan, ifosfamide, cyclophosphamide, melphalan, altretamine, dacarbazine, thiotepa, nimustine, chlorambucil, mitolactol, leucovorin, tretonin, exemestane, amino glutesimide, anagrelide, olaparib, navelbine, fadrazole, tamoxifen, toremifene, testolactone, anastrozole, letrozole, vorozol, bicalutamide, lomustine, vorinostat, entinostat, phenformin, metformin, talazoparib, and carmustine, without being limited thereto.

In one embodiment of the present invention, there is provided a pharmaceutical composition for preventing or treating cancer containing a sodium-glucose cotransporter-2 inhibitor or a pharmaceutically acceptable salt thereof as an active ingredient. In the pharmaceutical composition, the sodium-glucose cotransporter-2 inhibitor is any one or more selected from the group consisting of canagliflozin, dapagliflozin, empagliflozin, sotagliflozin, ipragliflozin, luseogliflozin, ertugliflozin, tofogliflozin, and remogliflozin. In the pharmaceutical composition, the inhibitor is any one or more selected from the group consisting of canagliflozin, dapagliflozin, and empagliflozin. In the pharmaceutical composition, the inhibitor is canagliflozin. The pharmaceutical composition for preventing or treating cancer further contains gossypol. The pharmaceutical composition for preventing or treating cancer further contains a biguanide-based compound as an active ingredient. In the pharmaceutical composition, the biguanide-based compound is any one or more selected from the group consisting of phenformin, metformin, and buformine. In the pharmaceutical composition, the biguanide-based compound is phenformin. In the pharmaceutical composition, the cancer comprises cancer stem cells.

In another embodiment of the present invention, there is provided a pharmaceutical composition for inhibiting cancer metastasis containing a sodium-glucose cotransporter-2 inhibitor or a pharmaceutically acceptable salt thereof as an active ingredient. In the pharmaceutical composition, the sodium-glucose cotransporter-2 inhibitor is any one or more selected from the group consisting of canagliflozin, dapagliflozin, empagliflozin, sotagliflozin, ipragliflozin, luseogliflozin, ertugliflozin, tofogliflozin, and remogliflozin. In the pharmaceutical composition, the inhibitor is canagliflozin. The pharmaceutical composition for inhibiting cancer metastasis further contains a biguanide-based compound as an active ingredient. In the pharmaceutical composition, the cancer comprises cancer stem cells.

In still another embodiment of the present invention, there is provided a food composition for preventing or ameliorating cancer containing a sodium-glucose cotransporter-2 inhibitor or a pharmaceutically acceptable salt thereof as an active ingredient. The food composition further contains gossypol. The food composition further contains a biguanide-based compound as an active ingredient. In the food composition, the cancer comprises cancer stem cells.

In yet another embodiment of the present invention, there is provided a pharmaceutical composition, containing a sodium-glucose cotransporter-2 inhibitor or a pharmaceutically acceptable salt thereof as an active ingredient, for use in a method for preventing or treating cancer.

In still yet another embodiment of the present invention, there is provided a pharmaceutical composition, containing a sodium-glucose cotransporter-2 inhibitor or a pharmaceutically acceptable salt thereof as an active ingredient, for use in a method for inhibiting cancer metastasis.

In a further embodiment of the present invention, there is provided a method for preventing or treating cancer, comprising a step of administering to a subject in need thereof a pharmaceutically effective amount of a sodium-glucose cotransporter-2 inhibitor or a pharmaceutically acceptable salt thereof.

In another further embodiment of the present invention, there is provided a method for inhibiting cancer metastasis, comprising a step of administering to a subject in need thereof a pharmaceutically effective amount of a sodium-glucose cotransporter-2 inhibitor or a pharmaceutically acceptable salt thereof.

Hereinafter, the present invention will be described in detail step by step.

### Advantageous Effects

The present invention is directed to a pharmaceutical composition for co-administration for preventing or treating cancer, containing, as an active ingredient, any one or more selected from the group consisting of an SGLT-2 inhibitor and gossypol, or derivatives or pharmaceutically acceptable salts thereof. The pharmaceutical composition of the present invention is expected to be widely used in the medical and health fields because the effect of killing cancer cells and/or cancer stem cells is significantly improved when the active ingredients are administered in combination compared to when the active ingredients are administered alone.

### BRIEF DESCRIPTION OF DRAWINGS

FIG. 1a shows the cancer cell killing effect when canagliflozin was administered alone or in combination with gossypol and/or phenformin to the lung cancer cell line A549 according to one example of the present invention.

FIG. 1b shows the cancer cell killing effect when metformin was administered alone or in combination with canagliflozin to the lung cancer cell line A549 according to one example of the present invention.

FIGS. 1c and 1d show the cancer cell killing effect when an SGLT-2 inhibitor (canagliflozin, dapagliflozin, or empagliflozin), gossypol, and phenformin were administered alone or in combination to the lung cancer cell line A549 according to one example of the present invention.

FIG. 2a shows the cancer cell killing effect when canagliflozin, gossypol, and phenformin were administered alone or in combination to the pancreatic cancer cell line Mia Paca-2 according to one example of the present invention.

FIGS. 2b and 2c show the cancer cell killing effect when an SGLT-2 inhibitor (canagliflozin, dapagliflozin, or empagliflozin), gossypol, and phenformin were administered alone or in combination to the pancreatic cancer cell line Mia Paca-2 according to one example of the present invention.

FIG. 3a shows the cancer cell killing effect when canagliflozin, gossypol, and phenformin were administered alone or in combination to the brain cancer cell line U87 according to one example of the present invention.

FIGS. 3b and 3c show the cancer cell killing effect when an SGLT-2 inhibitor (canagliflozin, dapagliflozin, or empagliflozin), gossypol, and phenformin were administered alone or in combination to the brain cancer cell line U87 according to one example of the present invention.

FIG. 4 shows the cancer cell killing effect when canagliflozin, gossypol, and/or phenformin were administered alone or in combination to the colon cancer cell line SW480 according to one example of the present invention.

FIGS. 5a to 5c show the ATP synthesis inhibitory effect of administration of canagliflozin, gossypol, and phenformin alone or in combination to lung cancer, pancreatic cancer, and brain cancer cell lines, respectively, according to one example of the present invention.

FIGS. 6a and 6b show the results of microscopic observation of the brain cancer cell line U87 after administration of canagliflozin, gossypol, and phenformin alone or in combination after spheroid culture of the brain cancer cell line U87 according to one example of the present invention, and show the changes in diameter of tumor spheroids following drug administration.

FIGS. 7a and 7b show the results of microscopic observation of the brain cancer cell line U87 after administration of dapagliflozin, gossypol, and phenformin alone or in combination after spheroid culture of the brain cancer cell line U87 according to one example of the present invention, and show the changes in diameter of tumor spheroids following drug administration.

FIGS. 8a and 8b show the results of microscopic observation of the brain cancer cell line U87 after administration of empagliflozin, gossypol, and phenformin alone or in combination after spheroid culture of the brain cancer cell line U87 according to one example of the present invention, and show the changes in diameter of tumor spheroids following drug administration.

FIG. 9 shows the results of microscopic observation of the brain cancer cell line U87 after administration of an SGLT-2 inhibitor (canagliflozin, dapagliflozin or empagliflozin) alone or in combination with gossypol and phenformin after spheroid culture of the brain cancer cell line U87 according to one example of the present invention.

FIG. 10 shows the results of measuring the oxygen consumption rate (OCR) during mitochondrial respiration after administration of an SGLT-2 inhibitor (canagliflozin, dapagliflozin or empagliflozin) alone or in combination with gossypol and phenformin to the lung cancer cell line A549 according to one example of the present invention.

### BEST MODE

One embodiment of the present invention is directed to a pharmaceutical composition for preventing or treating cancer containing a sodium-glucose cotransporter-2 inhibitor or a pharmaceutically acceptable salt thereof as an active ingredient.

Another embodiment of the present invention is directed to a pharmaceutical composition for inhibiting cancer metastasis containing a sodium-glucose cotransporter-2 inhibitor or a pharmaceutically acceptable salt thereof as an active ingredient.

Still another embodiment of the present invention is directed to a food composition for preventing or ameliorating cancer containing a sodium-glucose cotransporter-2 inhibitor or a pharmaceutically acceptable salt thereof as an active ingredient.

Yet another embodiment of the present invention is directed to a pharmaceutical composition, containing a sodium-glucose cotransporter-2 inhibitor or a pharmaceutically acceptable salt thereof as an active ingredient, for use in a method for preventing or treating cancer.

Still yet another embodiment of the present invention is directed to a pharmaceutical composition, containing a sodium-glucose cotransporter-2 inhibitor or a pharmaceutically acceptable salt thereof as an active ingredient, for use in a method for inhibiting cancer metastasis.

A further embodiment of the present invention is directed to a method for preventing or treating cancer, comprising a step of administering to a subject in need thereof an effective amount of a sodium-glucose cotransporter-2 inhibitor or a pharmaceutically acceptable salt thereof.

Another further embodiment of the present invention is directed to a method for inhibiting cancer metastasis, comprising a step of administering to a subject in need thereof an effective amount of a sodium-glucose cotransporter-2 inhibitor or a pharmaceutically acceptable salt thereof.

### MODE FOR INVENTION

Hereinafter, the present invention will be described in more detail by way of examples. These examples are only for illustrating the present invention in more detail, and it will be apparent to those skilled in the art that the scope of the present invention according to the subject matter of the present invention is not limited by these examples.

### Example 1: Culture of Cancer Cells

The present inventors obtained the human lung cancer cell line A549, the pancreatic cancer cell line Mia Paca-2, the brain cancer cell line U87, and the colon cancer cell line SW480 from the American Type Culture Collection (ATCC). Each of the cell lines was cultured according to the ATCC guidelines in 10% FBS-containing RPMI 1640, DMEM, or Eagle's MEM medium supplemented with 1% penicillin-streptomycin (Gibco) in a 5% CO₂ incubator (HERAcell 150i, Thermo Scientific, Waltham, MA, USA) at 37° C. and was used in the experiment.

### Example 2: Evaluation of Cancer Therapeutic Effect of Administration of Canagliflozin, Gossypol, and Biguanid-Based Compound Alone or in Combination

### 2.1 Therapeutic Effect on Lung Cancer Cell Line A549

### 2.1.1 Effect of Co-Administration of Canagliflozin, Gossypol, and Phenformin

The lung cancer cell line A549 cells prepared by the method of Example 1 were seeded into a 96-well plate at a concentration of 1×10⁴ cells/well and cultured overnight at 37° C. in a 5% CO₂ environment. Thereafter, the cells were treated with the drug canagliflozin at a concentration of 10 μM, 25 μM, or 50 μM alone or in combination with 5 μM gossypol and/or 10 μM phenformin, or treated with DMSO as a negative control, and then the cells were cultured for an additional 24 hours at 37° C. in a 5% CO₂ environment. Here, the drugs were administered simultaneously, and the specific drug treatment concentrations are shown in Table 1 below.

TABLE 1

| | Composition |
|---|---|
| Control | — |
| Ca10 | Canagliflozin 10 μM |
| Ca25 | Canagliflozin 25 μM |
| Ca50 | Canagliflozin 50 μM |
| G | Gossypol 5 μM |
| G + Ca10 | Gossypol 5 μM + Canagliflozin 10 μM |
| G + Ca25 | Gossypol 5 μM + Canagliflozin 25 μM |
| G + Ca50 | Gossypol 5 μM + Canagliflozin 50 μM |
| P | Phenformin 10 μM |
| P + Ca10 | Phenformin 10 μM + Canagliflozin 10 μM |
| P + Ca25 | Phenformin 10 μM + Canagliflozin 25 μM |
| P + Ca50 | Phenformin 10 μM + Canagliflozin 50 μM |
| G + P | Gossypol 5 μM + Phenformin 10 μM |
| G + P + Ca10 | Gossypol 5 μM + Phenformin 10 μM + |
| | Canagliflozin 10 μM |
| G + P + Ca25 | Gossypol 5 μM + Phenformin 10 μM + |
| | Canagliflozin 25 μM |
| G + P + Ca50 | Gossypol 5 μM + Phenformin 10 μM + |
| | Canagliflozin 50 μM |

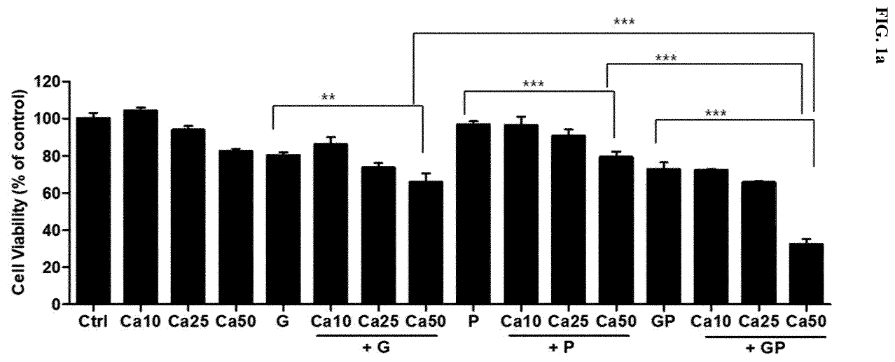

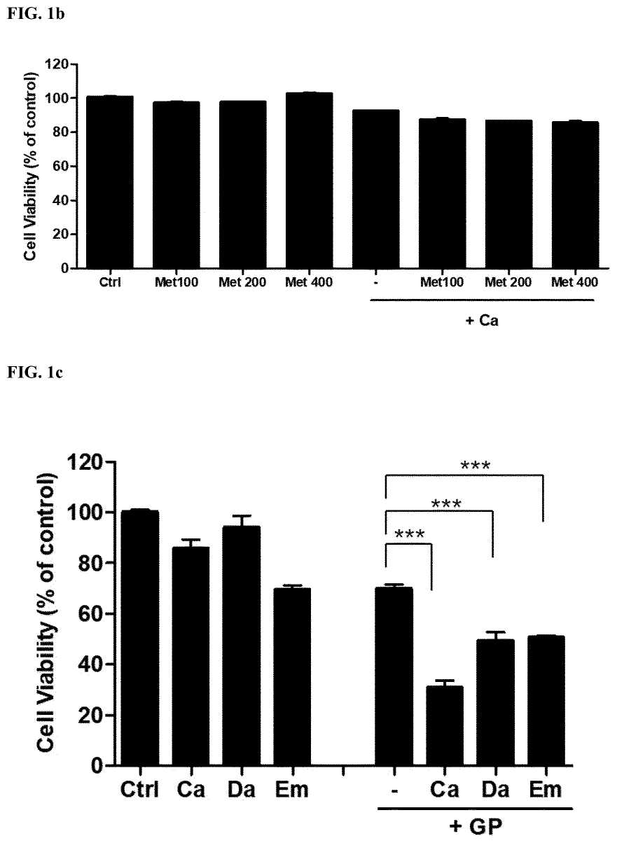

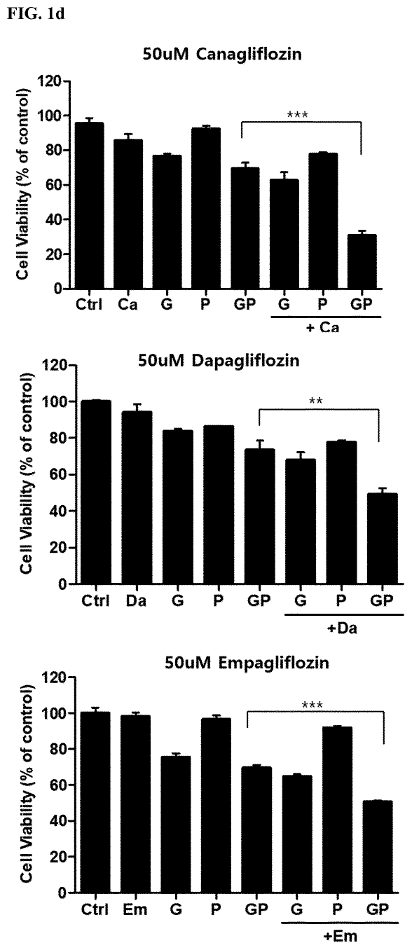

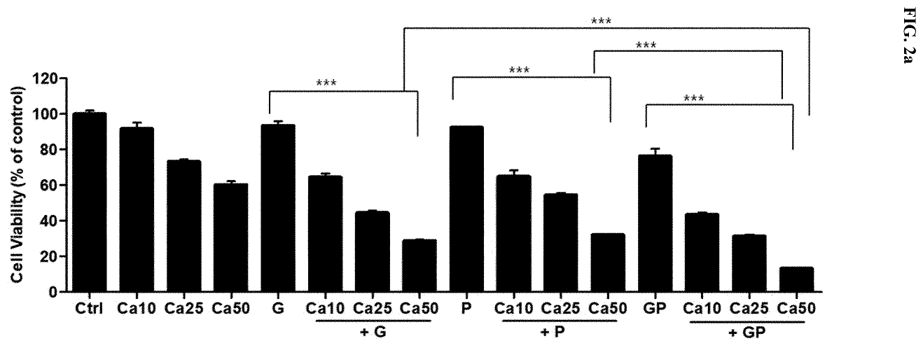

Thereafter, 100 μL of each cell culture and 10 μL of incubation buffer (CCK) were mixed together, placed in a 96-well plate, and incubated at room temperature for 1 hour, and then the absorbance was measured at a wavelength of 450 nm using a spectrophotometer (Synergy HTX Multi-Reader, BioTek, Winooski, VT, USA). At the same time, the number of cells in each sample was measured with a cell viability assay kit (Cell Counting Kit-8), and the measured value was calculated as a percentage of the number of cells in the negative control group to compare the number of cells between the samples. All experiments were repeated several times and the measured values were averaged. The results are shown in FIG. 1a. As a result of the experiment, it was confirmed that, compared to when 10 μM canagliflozin was administered alone to the lung cancer cell line, the cancer cell killing effect increased by about 40% when 10 μM canagliflozin was administered in combination with gossypol, and increased by about 25% when 10 μM canagliflozin was administered in combination with phenformin, and increased by 50% (the highest increase) when 10 UM canagliflozin was administered in combination with gossypol and phenformin. In addition, it was confirmed that, compared to when 25 μM canagliflozin was administered alone to the lung cancer cell line, the cancer cell killing effect increased by about 45% when 25 μM canagliflozin was administered in combination with gossypol, and increased by about 33% when 25 μM canagliflozin was administered in combination with phenformin, and increased by about 53% when 25 μM canagliflozin was administered in combination with gossypol and phenformin. In addition, it was confirmed that, compared to when 50 μM canagliflozin was administered alone to the lung cancer cell line, the cancer cell killing effect increased by about 53% when 50 μM canagliflozin was administered in combination with gossypol, and increased by about 50% when 50 μM canagliflozin was administered in combination with phenformin, and increased by about 68% when 50 M canagliflozin was administered in combination with gossypol and phenformin. Thereby, it was confirmed that the cancer therapeutic effect, that is, the cancer killing effect, significantly increased by 50% or more when canagliflozin was administered in combination with gossypol and phenformin compared to when canagliflozin was administered alone. Also, it could be confirmed that the higher the concentration within the tested range, the degree of death of cancer cells was higher (see FIG. 1a). It could also be confirmed that the cancer therapeutic effect increased when canagliflozin was administered in combination with both gossypol and phenformin compared to when canagliflozin was administered in combination with gossypol or phenformin. The numerical values on the drawings according to all examples below are expressed as mean±standard error of the mean (SEM), and * indicates statistical significance (**P<0.01, ***P<0.001).

### 2.1.2 Effects of Co-Administration of Canagliflozin and Metformin

To further compare and evaluate the effect of co-administration of metformin, not phenformin, as a biguanide-based compound, and canagliflozin, the drugs were administered in the concentration range shown in Table 2 below and then the cell viability of the lung cancer cell line was measured.

TABLE 2

| | Composition |
|---|---|
| control | — |
| met100 | Metformin 100 μM |
| met200 | Metformin 200 μM |
| met400 | Metformin 400 μM |
| Ca50 | Canagliflozin 50 μM |
| Ca50 + met100 | Canagliflozin 50 μM + Metformin 100 μM |
| Ca50 + met200 | Canagliflozin 50 μM + Metformin 200 μM |
| Ca50 + met400 | Canagliflozin 50 μM + Metformin 400 μM |

As a result of the experiment, it was confirmed that, when metformin in the concentration range of 100 to 400 μM was administered in combination with 50 μM of canagliflozin, there was no significant difference in the degree of cancer cell death. Thereby, it could be confirmed that the synergistic effect shown when administered in combination with phenformin did not appear when administered in combination with metformin, even though phenformin and metformin are all biguanide-based compounds (see FIG. 1b).

### 2.1.3 Comparison of Co-Administration Effects According to Types of SGLT-2 Inhibitors

The present inventors sought to evaluate the cancer cell killing effect when each of various SGLT-2 inhibitors was administered in combination with gossypol and/or phenformin to the lung cancer cell line prepared by the method of Example 1. In particular, in order to clearly compare the synergistic effects on the same line when administered in combination with different types of SGLT-2 inhibitors, experiments were performed with all combinations possible in terms of probability. Here, the concentration of the inhibitor was fixed at 50 μM, and gossypol at a concentration of 5 μM and/or phenformin at a concentration of 10 μM were administered alone or in combination with the inhibitor.

The experimental results are shown in FIGS. 1c and 1d. Overall, it was confirmed that all of the types of SGLT-2 inhibitors exhibited a synergistic effect when administered in combination with gossypol and phenformin, and among the SGLT-2 inhibitors, canagliflozin exhibited the highest synergistic effect (see FIG. 1c). More specifically, an additional experiment was conducted to individually evaluate the effect of co-administration of the SGLT-2 inhibitor and each of gossypol and phenformin and the effect of co-administration of the SGLT-2 inhibitor and the two drugs. The above experiment was also performed according to the method described in Example 2, and the results after drug treatment are shown in FIG. 1d. Repeated experiments showed that the degree of cancer cell death was greatest when administered when the SGLT-2 inhibitor was administered in combination with gossypol and phenformin. In addition, a significant effect could also be found when the SGLT-2 inhibitor and gossypol were administered in combination compared to when each drug was administered alone.

### 2.2 Therapeutic Effect on Pancreatic Cancer Cell Line Mia Paca-2

According to the methods of Example 1 and Example 2, the pancreatic cancer cell line Mia Paca-2 cells were cultured, and then each drug was administered to the cells, followed by analysis of the cell viability.

2.2.1 Effects of Co-Administration of Canagliflozin with Gossypol and/or Phenformin

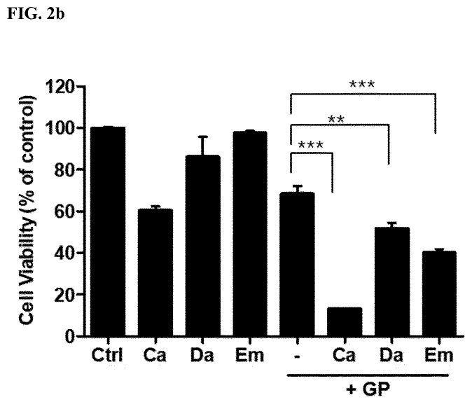

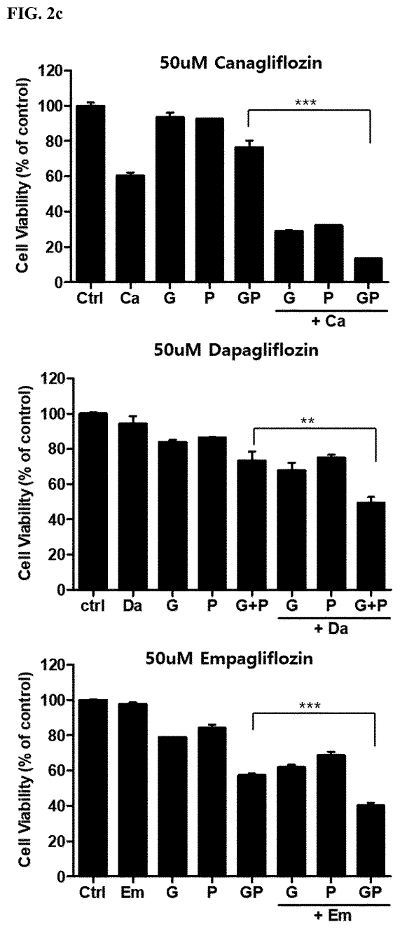

As a result of the experiment, a tendency similar to that in the lung cancer cell line was seen in the pancreatic cancer cell line (see FIG. 2a). It could be confirmed that, compared to when canagliflozin was administered alone, the cancer cell killing effect increased by about 40 to 50% when canagliflozin was administered in combination with gossypol and phenformin, even though the effect differed slightly depending on the concentration of canagliflozin. In addition, it could be confirmed that the cancer cell killing effect increased as the concentration increased within the tested concentration range. However, it could be confirmed that, at the same treatment concentration, the cancer cell killing effect was more significant against the pancreatic cancer cell line than the lung cancer cell line (see FIGS. 1a and 2a).

### 2.2.2 Comparison of Co-Administration Effects According to Types of SGLT-2 Inhibitors

As a result of the experiment, a tendency similar to that in the lung cancer cell line could be seen in the pancreatic cancer cell line (see FIG. 2b). It was confirmed that, compared to when the SGLT-2 inhibitor was administered alone, the cancer therapeutic effect increased significantly when the SGLT-2 inhibitor was administered in combination with gossypol and phenformin, and in particular, among the SGLT-2 inhibitors, canagliflozin was more effective. In addition, it could be confirmed that the cancer cell viability of the pancreatic cancer cell line was lower when the three drugs were administered in combination, as in the lung cancer cell line, indicating that the co-administration exhibited a synergistic effect (see FIG. 2c). However, what is important to note here is the effect of co-administration of canagliflozin and gossypol. As a result of the experiment, it can be seen that a synergistic effect on cancer treatment can be expected when canagliflozin is administered in combination with gossypol or with phenformin.

### 2.3 Therapeutic Effect on Brain Cancer Cell Line U87

According to the methods of Example 1 and Example 2, the brain cancer cell line U87 cells were cultured, and then each drug was administered to the cells, followed by analysis of the cell viability.

2.3.1 Effect of Co-Administration of Canagliflozin with Gossypol and/or Phenformin

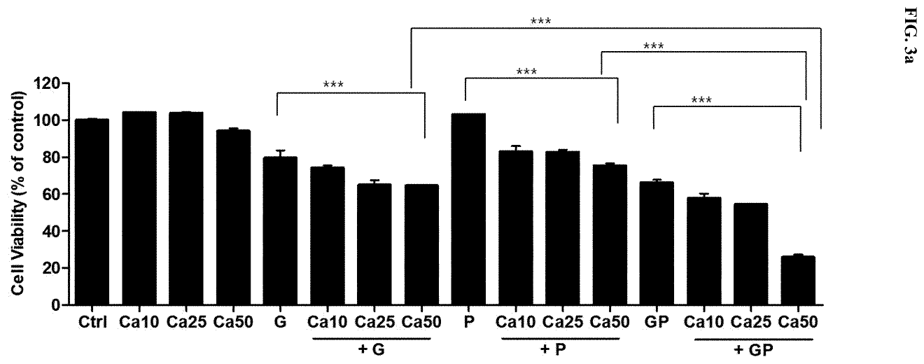

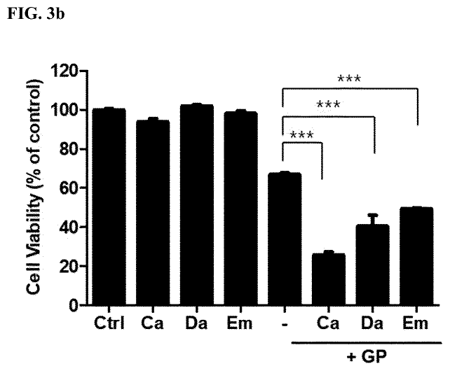

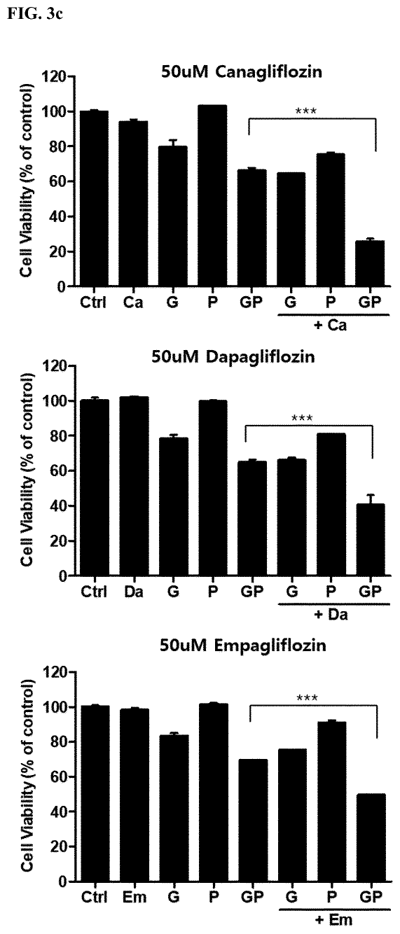

As a result of the experiment, it was confirmed that the brain cancer cell line showed a similar tendency to that of the lung cancer and pancreatic cancer cell lines (see FIG. 3a). It can be confirmed that the cancer killing effect was improved when canagliflozin was administered in combination with gossypol and phenformin compared to when canagliflozin was administered alone. More specifically, it could be confirmed that, at 50 μM in the concentration range of canagliflozin tested, the therapeutic effect increased by almost 60% due to the synergistic effect of co-administration of canagliflozin with gossypol and phenformin.

### 2.3.2 Comparison of Co-Administration Effects According to Types of SGLT-2 Inhibitors

As a result of the experiment, a tendency similar to that in the pancreatic cancer and lung cancer cell lines could be seen (see FIG. 3b). In addition, it was confirmed that, even in the brain cancer cell line, the cancer therapeutic effect increased significantly when the SGLT-2 inhibitor was administered in combination with gossypol and phenformin compared to when the SGLT-2 inhibitor was administered alone, and in particular, among the SGLT-2 inhibitors, canagliflozin exhibited a better effect (FIG. 3c).

### 2.4 Therapeutic Effect on Colon Cancer Cell Line SW480

According to the methods of Example 1 and Example 2, the colon cancer cell line SW480 cells were cultured, and then each drug was administered to the cells, followed by analysis of the cell viability. The results are shown in FIG. 4, and a significantly improved effect can be seen when administering canagliflozin and gossypol in combination and when administering canagliflozin in combination with gossypol and phenformin.

Taking the results of Example 2 together, it can be confirmed that various cancer cell lines show the same tendency, indicating that the synergistic effect of co-administration appears regardless of the type of cancer. In particular, it is considered worth noting that the rate of increase in effect when canagliflozin is used in combination with gossypol and phenformin is the highest in the case of pancreatic cancer compared to lung cancer and brain cancer regardless of canagliflozin concentration (see FIGS. 1a, 2a, and 3a).

### Example 3: Evaluation of ATP Synthesis Inhibitory Effect when Administered Alone or in Combination in Various Cancer Cell Lines

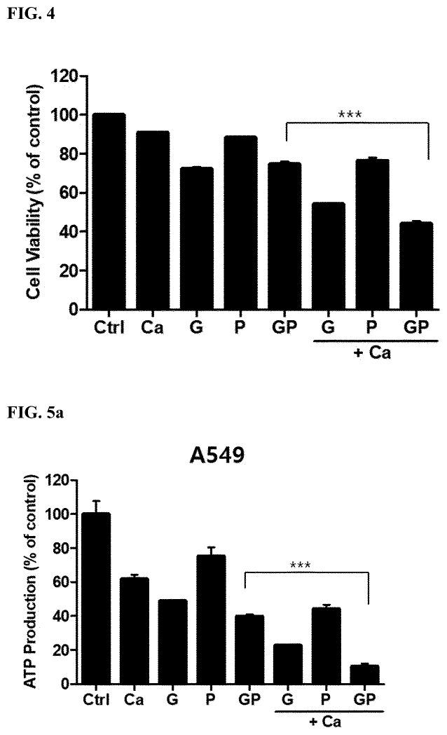

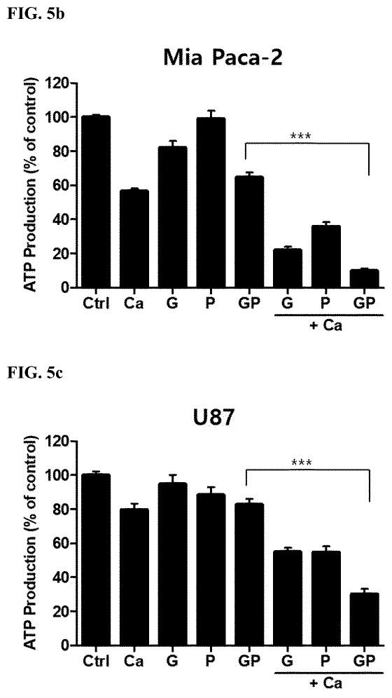

The lung cancer cell line A549 cells, pancreatic cancer cell line Mia Paca-2 cells, or brain cancer cell line U87 cells, prepared according to the method of Example 1, were seeded into 96-well plates at a concentration of 1×10⁴ cells/well and cultured overnight at 37° C. in a 5% CO₂ environment. Thereafter, the cells were treated with canagliflozin, gossypol, and/or phenformin at concentrations of 50 μM, 5 μM, and/or 10 μM, or treated with DMSO as a negative control, and then cultured for an additional 24 hours at 37° C. in a 5% CO₂ environment. After removing the cell medium, the cells were treated with 50 μL/well of detergent using the luminescent ATP detection assay kit (Abcam, USA), incubated at room temperature for 30 minutes, treated with 50 μL/well of substrate solution, and then incubated at room temperature for an additional 30 minutes. Next, the luminescence was measured using the Synergy HTX Multi-Reader (BioTek). The measured value was calculated as a percentage of the cell luminescence of the negative control group to compare the cell luminescence between the samples. All experiments were repeated several times and the measured values were averaged. The results are shown in FIGS. 5a to 5c.

A low measurement of cell luminescence means that ATP synthesis in mitochondria is inhibited, and it can be seen that this inhibition of ATP synthesis leads to inhibition of proliferation of cancer cells. As a result of the experiment, it can be confirmed that the ATP synthesis inhibitory effect in the lung cancer cells and pancreatic cancer cells was generally higher than that in the brain cancer cells, and the ATP synthesis inhibitory effect of the combination of canagliflozin and gossypol was significantly higher than that in the group to which canagliflozin or gossypol was administered alone (see FIGS. 5a to 5c). However, although it could be confirmed that the combination of canagliflozin and gossypol had a relatively low effect on brain cancer, co-administration of canagliflozin, gossypol and phenformin can be expected to exhibit a therapeutic effect even on brain cancer.

Taking the ATP assay results together, the degree to which ATP synthesis in mitochondria is inhibited is greater in the group to which the drugs are administered in combination than in the group to which each drug is administered alone, which suggests that the cancer therapeutic effect can significantly increase when the drugs are administered in combination.

### Example 4: Examination of Change in Spheroid Diameter when Administered Alone or in Combination

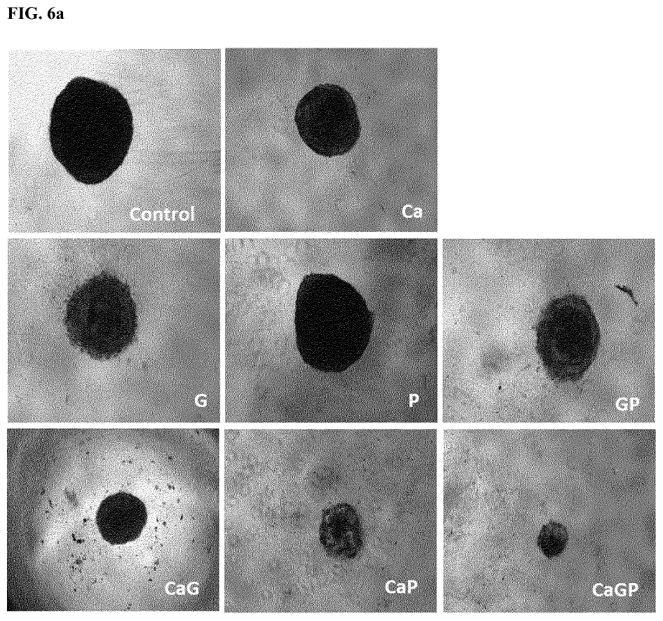

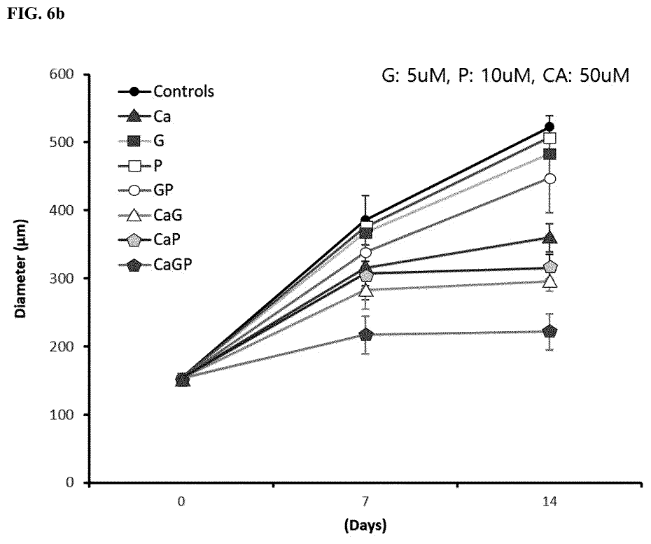

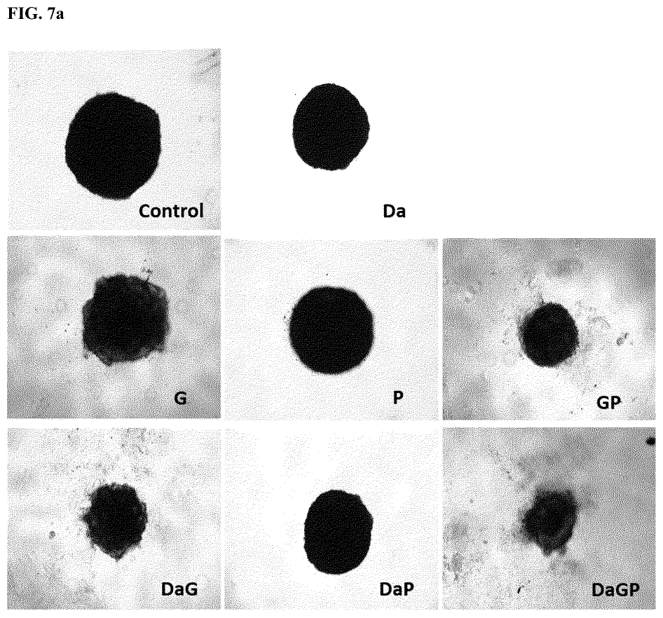

The U87 cell line cells prepared according to the method of Example 1 were seeded into a 96-well plate at a density of 1.0×10² cells/well in DMEM media containing 2% Matrigel (Corning, NY, USA), and stabilized in a 5% CO₂ incubator at 37° C. for 3 days to form tumor spheroids. After stabilization, the cells were cultured in a 5% CO₂ incubator at 37° C. for 14 days. During the culturing, gossypol, phenformin, canagliflozin, dapagluflozin, and empagluflozin at the drug concentrations shown in Tables 3 to 5 described in Comparative Examples 1 to 3 below were administered alone or in combination to the cells at intervals of 2 to 3 days. In order to examine the shape and size of the tumor spheroids of the U87 cell line before and after drug treatment, the obtained cell cultures were observed under a microscope (Leica DMLB, Leica) and photographed with a digital camera (DP70 Digital Microscope Camera; Olympus), and the photographs are shown in FIGS. 6a, 7a and 8a. In addition, the change in diameter of the tumor spheroids by administration of each drug combination is graphically shown in FIGS. 6b, 7b and 8b. Referring to FIGS. 6b, 7b and 8b, the rate of change in diameter of the spheroid in the graph was calculated as a percentage based on the change in the average diameter after administration of each drug compared to the average diameter of tumor spheroid cells seeded in the 96-well plate.

4.1 Evaluation of Co-Administration Effect according to the Type of SGLT-2 Inhibitor

### Comparative Example 1

TABLE 3

| | Composition |
|---|---|
| Control | — |
| Ca | Canagliflozin 50 μM |
| G | Gossypol 5 μM |
| P | Phenformin 10 μM |
| GP | Gossypol 5 μM + Phenformin 10 μM |
| CaG | Canagliflozin 50 μM + Gossypol 5 μM |
| CaP | Canagliflozin 50 μM + Phenformin 10 μM |
| CaGP | Canagliflozin 50 μM + Gossypol 5 μM + Phenformin 10 μM |

As shown in the cell photographs of FIG. 6a, although the size of the tumor spheroid cells decreased even when canagliflozin, gossypol, or phenformin was administered alone, the size of tumor spheroid cells decreased to a greater extent when a combination of canagliflozin and gossypol or a combination of canagliflozin and phenformin was administered, and the size of the tumor spheroid cells was significantly reduced when a combination of canagliflozin, gossypol and phenformin was administered.

Moreover, as shown in FIG. 6b, which graphically shows numerical values converted clearly from the size observed under a microscope, it can be seen that the diameter of the tumor spheroid cells was significantly reduced when treated with a combination of the drugs compared to when each drug was administered alone.

### Comparative Example 2

TABLE 4

| | Composition |
|---|---|
| Control | — |
| Da | Dapagliflozin 75 μM |
| G | Gossypol 5 μM |
| P | Phenformin 10 μM |
| GP | Gossypol 5 μM + Phenformin 10 μM |
| DaG | Dapagliflozin 75 μM + Gossypol 5 μM |
| DaP | Dapagliflozin 75 μM + Phenformin 10 μM |
| DaGP | Dapagliflozin 75 μM + Gossypol 5 μM + Phenformin 10 μM |

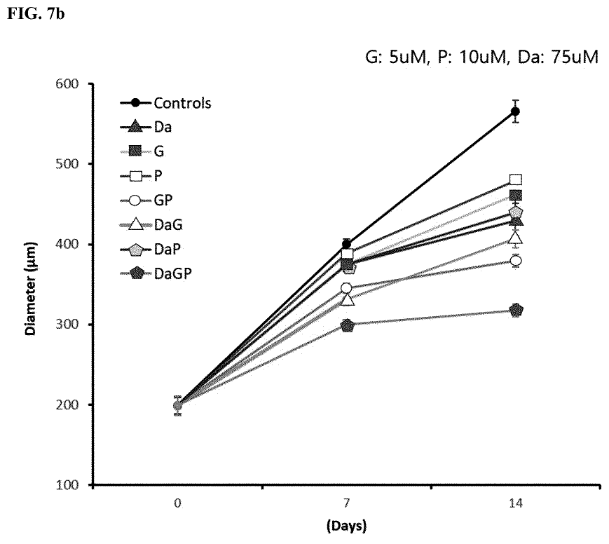

As shown in the cell photographs of FIG. 7a, although the size of the tumor spheroid cells decreased even when dapagliflozin, gossypol, or phenformin was administered alone, the size of the tumor spheroid cells decreased to a greater extent when a combination of dapagliflozin and gossypol, a combination of dapagliflozin and phenformin, or a combination of gossypol and phenformin was administered, and the size of the tumor spheroid cells was most significantly reduced when a combination of dapagliflozin, gossypol and phenformin was administered.

As shown in FIG. 7b, which graphically shows clearly converted numerical values, even in the experiment conducted using dapagliflozin instead of canagliflozin, it can be seen that the diameter of the tumor spheroid cells was significantly reduced when treated with a combination of the drugs compared to when each drug was administered alone.

### Comparative Example 3

TABLE 5

| | Composition |
|---|---|
| Control | — |
| Em | Empagliflozin 75 μM |
| G | Gossypol 5 μM |
| P | Phenformin 10 μM |
| GP | Gossypol 5 μM + Phenformin 10 μM |
| EmG | Empagliflozin 75 μM + Gossypol 5 μM |
| EmP | Empagliflozin 75 μM + Phenformin 10 μM |
| EmGP | Empagliflozin 75 μM + Gossypol 5 μM + Phenformin 10 μM |

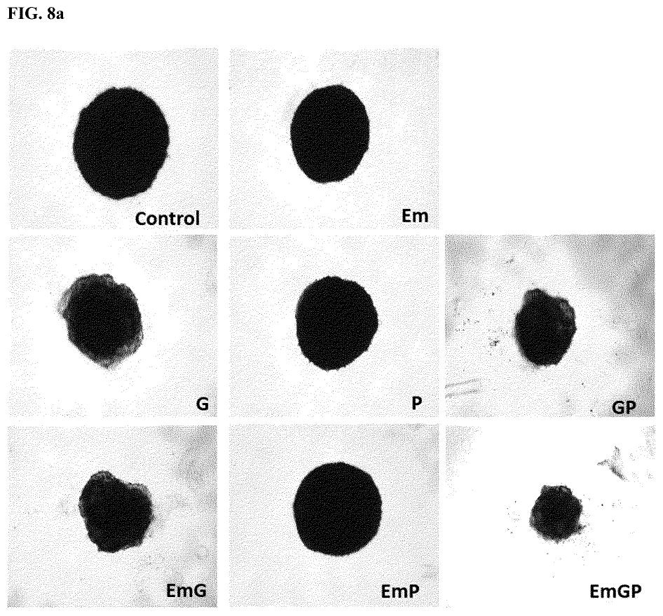

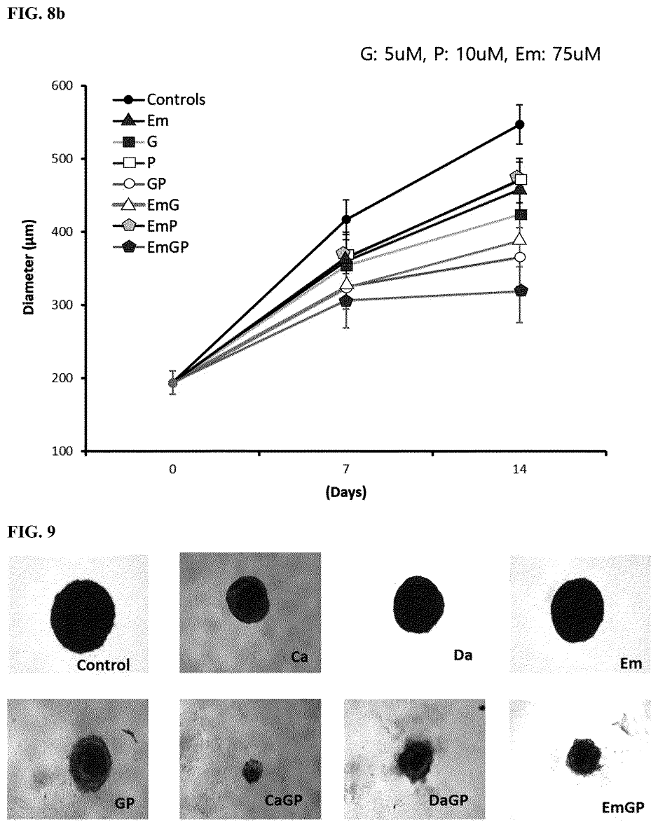

As shown in the cell photographs of FIG. 8a, although the size of the tumor spheroid cells decreased even when empagliflozin, gossypol, or phenformin was administered alone, the size of the tumor spheroid cells decreased to a greater extent when a combination of empagliflozin and gossypol, or a combination of gossypol and phenformin was administered, and the size of the tumor spheroid cells was most significantly reduced when a combination of empagliflozin, gossypol and phenformin was administered.

As shown in FIG. 8b, which graphically shows clearly converted numerical values, even in the experiment conducted using empagliflozin instead of canagliflozin, it can be seen that the diameter of the tumor spheroid cells was significantly reduced when treated with a combination of the drugs compared to when each drug was administered alone. In addition, as shown in cell photographs of FIG. 9, although the size of the cells decreased compared to that in the control group even when SGLT-2 inhibitors (canagliflozin, dapagliflozin, and empagliflozin) were administered alone, the size of the cells decreased when each SGLT-2 inhibitor was administered in combination with gossypol and phenformin more than when each SGLT-2 inhibitor was administered alone. Among the SGLT-2 inhibitors, canagliflozin showed the most significant reduction in the cell size.

### 4.2 Interpretation of Results

Through the experiments of Comparative Examples 1 to 3, it was confirmed that the size of tumor spheroids was significantly reduced when treated with a combination of the SGLT-2 inhibitor, gossypol, and phenformin in the present invention compared to when treated with each of them alone, which means that the combination of the SGLT-2 inhibitor, gossypol, and phenformin can effectively inhibit the proliferation and metastasis of brain cancer stem cells. Thus, it can further be seen that the combination can have a significantly improved effect on the treatment of brain cancer.

### Example 5: Comparison of Oxygen Consumption Rate (OCR) During Mitochondrial Respiration Between Single Administration and Co-Administration

In order to measure mitochondrial respiration, which is the main energy metabolic process of cells, the present inventors analyzed cellular oxygen consumption rate (OCR) by measuring intracellular oxygen concentration. The oxygen consumption rate that occurs during energy metabolism can be a measure of how actively cancer cell division occurs during the metabolic process of cancer cells.

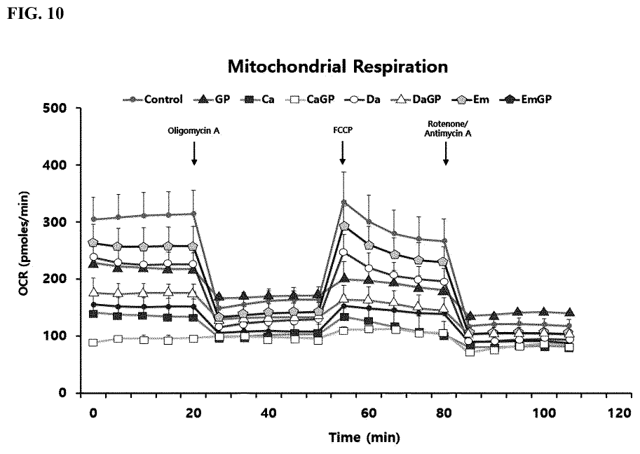

1 mL of XF Calibrant buffer (Agilent Technologies) was added to a sensor cartridge (Agilent Technologies, Palo Alto, CA, USA) and incubated in a non-CO₂ incubator overnight at 37° C. The lung cancer line A549 cells were seeded into the XF24 cell culture microplate (Agilent Technologies) at a density of 7×10⁴ cells/well, and then incubated in a 5% CO₂ incubator at 37° C. for 24 hours. Next, 5 μM gossypol, 10 μM phenformin, and 50 μM canagliflozin, dapagluflozin, or empagluflozin were administered alone or in combination to the cells, followed by incubation in a 5% CO₂ incubator at 37° C. overnight. XF base media (pH 7.4, Agilent Technologies) was added to the drug-administered microplate at 500 μL/well, followed by incubation in a non-CO₂ incubator at 37° C. for 1 hour. 1.5 μM oligomycin (Agilent Technologies) was added to port A of the sensor cartridge, 2.0 μM FCCP (Agilent Technologies) was added to port B, and 0.5 μM rotenone/antimycin A (Agilent Technologies) was added to port C. In addition, the XF24 microplate was placed in the Agilent seahorse XF24 (Agilent Technologies) instrument, and OCR was measured. The results are shown in FIG. 10. Here, the measured values were compared with percentage values based on picomoles per minute (pM/min).

As shown in FIG. 10, it can be could be confirmed that the oxygen consumption rate was lowest when a combination of canagliflozin, gossypol and phenformin was administered, and that the oxygen consumption rate was also low when dapagliflozin or empagluflozin as the SGLT-2 inhibitor was administered. A low oxygen consumption rate means that the cancer therapeutic effect is good.

Therefore, as confirmed in Examples 1 to 5, when the SGLT-2 inhibitor, gossypol, and biguanide-based compound of the present invention were administered in combination, they exhibited a very good cancer cell killing effect on various cancer types. Preferably, when a pharmaceutical composition containing, as active ingredients, the SGLT-2 inhibitor, gossypol, and phenformin, more preferably canagliflozin, gossypol, and phenformin, was administered, the cancer cell killing effect was more significant. In addition, since the pharmaceutical composition was found to exhibit the effect of inhibiting the proliferation and metastasis of brain cancer stem cells, it can be applied to the treatment of not only cancer cells but also cancer stem cells, and thus is expected to be widely used in the field of anticancer therapy.

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

### INDUSTRIAL APPLICABILITY

The composition of the present invention is applicable to various cancer types, and the effect of killing cancer cells and/or cancer stem cells is significantly improved when the active ingredients of the composition are administered in combination compared to when the active ingredients are administered alone. Thus, the composition is capable of effectively preventing, ameliorating or treating cancer.

1. A pharmaceutical composition for preventing or treating cancer containing gossypol, a biguanide-based compound, and a sodium-glucose cotransporter-2 inhibitor or a pharmaceutically acceptable salt thereof as an active ingredient.

2. The pharmaceutical composition of claim 1, wherein the sodium-glucose cotransporter-2 inhibitor is any one or more selected from the group consisting of canagliflozin, dapagliflozin, empagliflozin, sotagliflozin, ipragliflozin, luseogliflozin, ertugliflozin, tofogliflozin, and remogliflozin.

3. The pharmaceutical composition of claim 2, wherein the glucose cotransporter-2 inhibitor is any one or more selected from the group consisting of canagliflozin, dapagliflozin, and empagliflozin.

4. (canceled)

5. (canceled)

6. (canceled)

7. The pharmaceutical composition of claim 16, wherein the biguanide-based compound is any one or more selected from the group consisting of phenformin, metformin, and buformine.

8. The pharmaceutical composition of claim 7, wherein the biguanide-based compound is phenformin.

9. The pharmaceutical composition of claim 1, wherein the cancer comprises cancer stem cells.

10. The pharmaceutical composition of claim 1, wherein the treating comprises suppressing an increase in the number of cancer cells, including cancer stem cells, or inhibiting quantitative proliferation of the cancer cells, killing the cells, or reducing or maintaining the size of cancer tissue containing cancer stem cells, or inhibiting the development of new blood vessels in cancer tissue containing cancer stem cells.

11. The pharmaceutical composition of claim 1, wherein the treating comprises inhibiting cancer metastasis.

12. (canceled)

13. (canceled)

14. (canceled)

15. (canceled)

16. (canceled)

17. A food composition for preventing or ameliorating cancer containing gossypol, a biquanide-based compound, and a sodium-glucose cotransporter-2 inhibitor or a pharmaceutically acceptable salt thereof as an active ingredient.

18. (canceled)

19. (canceled)

20. The food composition of claim 17, wherein the cancer comprises cancer stem cells.

21. The food composition of claim 17, wherein the ameliorating comprises suppressing an increase in the number of cancer cells, including cancer stem cells, or inhibiting quantitative proliferation of the cancer cells, killing the cells, or reducing or maintaining the size of cancer tissue containing cancer stem cells, or inhibiting the development of new blood vessels in cancer tissue containing cancer stem cells.

22. (canceled)

23. (canceled)

24. A method for preventing or treating cancer, comprising a step of administering to a subject in need thereof a pharmaceutically effective amount of gossypol, a biguanide-based compound, and a sodium-glucose cotransporter-2 inhibitor or a pharmaceutically acceptable salt thereof.

25. The method for preventing or treating cancer of claim 1, wherein the treating comprises inhibiting cancer metastasis.
